(19) United States
(12) Patent Application Publication
(10) Pub. No.: US 2023/0173007 A1
(43) Pub. Date: Jun. 8, 2023

(54) Natural Cannabinoid Extraction Process

(71) Applicant: David Neal, Chicago, IL (US)

(72) Inventor: David Neal, Chicago, IL (US)

(21) Appl. No.: 18/062,358

(22) Filed: Dec. 6, 2022

Related U.S. Application Data
(60) Provisional application No. 63/286,614, filed on Dec. 7, 2021.

(51) Int. Cl. A61K 36/185

(52) U.S. Cl. CPC A61K 36/185; A61K 2236/31; A61K 2236/37; A61K 2236/331

(57) ABSTRACT

The present invention relates to a novel natural extraction method for cannabinoid without using any additives, alcohol, or butane. The method is configured to extract sap from the cannabis via high-pressure, heated water rather than butane or alcohol. More specifically, natural ingredients such as calcium, magnesium, organic citrus, and mint are used and mixed together with cannabis strain in a coffee filter or cheese cloth filter for heating in water. The water, having the ingredients positioned and packaged similar to a tea bag therein, is either boiled for two hours in a crockpot or for seven minutes in a pressure cooker for providing sap and bud of cannabis. In some embodiments, the cannabis strain is dried and grinded before mixing with other ingredients. The method offers a way to extract cannabinoids swiftly and easily for making cannabis edibles.

### CROSS-REFERENCE TO RELATED APPLICATION

The present application claims priority to, and the benefit of, U.S. Provisional Application No. 63/286,614, which was filed on December 7th, 2021, and is incorporated herein by reference in its entirety.

### FIELD OF THE INVENTION

The present invention generally relates to the field of cannabis-related products and cannabis extraction methodologies. More specifically, the present invention relates to a novel method of extracting cannabidiol from cannabis strain without using butane, alcohol, and additive preservatives. The method includes several different natural ingredients, such as calcium, magnesium, organic citrus, and mint, and more, and offers a way to extract cannabinoids swiftly and easily for making cannabis edibles. Accordingly, the present disclosure makes specific reference thereto. Nonetheless, it is to be appreciated that aspects of the present invention are also equally applicable to other like applications, devices, and methods of manufacture.

### BACKGROUND

By way of background, Cannabis, also known as marijuana among other names, is a psychoactive drug from the Cannabis plant. Also, Cannabis refers to a group of three plants with psychoactive properties, known as Cannabis sativa, Cannabis indica, and Cannabis ruderalis. Cannabis has major compounds such as delta-9-tetrahydrocannabinol and cannabidiol, which have opposing effects. For producing cannabis for use by consumers, a process of cannabis extraction is used by producers. The main objective of cannabis extraction is to produce high-quality cannabidiol (CBD) and Tetrahydrocannabinol (THC) products. The purpose of cannabis extraction is to isolate the various compounds featured within the plant and use them to enhance the quality of cannabis for consumers.

Currently, there are many methods of extracting cannabis, but all of the methods use alcohol or butane to separate the oils (rich with THC and CBD) into a usable form. The simplest method is to make an alcohol-based tincture, where cannabis is soaked in alcohol at room temperature for weeks. Most methods for cannabis extraction use liquid solvent extraction which uses liquid solvents such as alcohol, to extract the CBD and other desired properties from the hemp. Alcohol such as isopropyl alcohol and grain alcohol are commonly used in cannabis extraction.

Use of butane and alcohol require precise management of temperature and butane is extremely dangerous for individuals and requires proper precautions to be used while using butane. Further, if butane is not properly treated it can get mixed into the CBD and THC, and still further, inhaling inadequately purged butane can cause a severe lung condition.

If extraction process using hydrocarbons (butane, propane, and more) is not carried out properly, the process can be dangerous. Special equipment and working conditions are required for extracting cannabis oil, which is costly and time consuming. Therefore, individuals desire a cannabinoid extraction process that does not utilize butane or alcohol.

Therefore, there exists a long-felt need in the art for a cannabinoid extraction process that does not utilize butane or alcohol. There is also a long-felt need in the art for a cannabinoid extraction process that includes natural ingredients. Additionally, there is a long-felt need in the art for a cannabinoid extraction process that is easy to perform and is cost-effective. Moreover, there is a long-felt need in the art for a cannabinoid extraction process that eliminates mixing of butane or alcohol in CBD and THC. Further, there is a long-felt need in the art for a cannabinoid extraction process that provides the purest form of cannabinoid to consumers. Furthermore, there is a long-felt need in the art for a cannabinoid extraction process that can be used for both commercial and personal production of cannabis oils. Finally, there is a long-felt need in the art for a cannabinoid extraction process that offers a way to extract cannabinoids swiftly and easily for making cannabis edibles.

The subject matter disclosed and claimed herein, in one embodiment thereof, comprises a cannabinoid extraction method from cannabis without using butane or alcohol. The method is natural, organic, easy and quick and comprises the steps of, in one exemplary arrangement, providing about 1.0 gram of hemp or cannabis strain; providing from about 2.0 grams to about 3.0 grams calcium powder; providing from about 2.0 grams to about 3.0 grams magnesium powder; providing from about 4.0 grams to about 5.0 grams citrus; providing from about 12.0 grams to about 16.0 grams mint leaves; and, providing about 250 ml of filtered water. The steps further comprise mixing the calcium powder, magnesium powder, citrus, and mint leaves (and may also comprise natural preservatives); storing the mixture in a 4″ x 4″ cheese cloth piece; tying the cheese cloth piece using a hemp string; placing the cheese cloth piece like a tea bag in the filtered water placed inside a crockpot; boiling the water in the crackpot for 2 hours or boiling the water in a pressure cooker for 7 minutes; and extracting sap from the leaves and bud of cannabis for providing THC and CBD.

In this manner, the natural cannabinoid extraction process of the present invention accomplishes all of the forgoing objectives and provides users with a cannabinoid extraction process and method that does not utilize butane or alcohol. The process is easy and safe to perform and offers a way to extract cannabinoids swiftly and easily for making cannabis edibles. The use of natural ingredients does not pose a threat of any additives and maintains a pure form of the cannabinoids.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a method of extracting sap from cannabis without using butane or alcohol. The method comprising the steps of providing one gram of cannabis strain; providing ½ teaspoon of calcium powder, ½ teaspoon of magnesium powder, 1 teaspoon of citrus, 1 tablespoon of mint leaves, and 1 cup of filtered water; mixing the calcium powder, magnesium powder, citrus and mint leaves; storing the mixture in a 4″ x 4″ cheese cloth piece; tying the cheese cloth piece using a hemp string; placing the cheese cloth piece like a tea bag in the filtered water placed inside a crackpot; boiling the water in the crackpot for 2 hours; and extracting sap of leaves and bud of cannabis for providing THC and CBD.

The subject matter disclosed and claimed herein, in one embodiment thereof, comprises a method of extracting sap from cannabis without using butane or alcohol. The method comprising the steps of providing one gram of cannabis strain; providing ½ teaspoon of calcium powder, ½ teaspoon of magnesium powder, 1 teaspoon of citrus, 1 tablespoon of mint leaves, and 1 cup of filtered water; mixing the calcium powder, magnesium powder, citrus and mint leaves; storing the mixture in a 4″ x 4″ cheese cloth piece; tying the cheese cloth piece using a hemp string; placing the cheese cloth piece like a tea bag in the filtered water placed inside a pressure cooker; boiling the water in the pressure cooker for 7 minutes; and extracting sap of leaves and bud of cannabis for providing THC and CBD.

In yet another embodiment, the cannabis strain is selected from one or combination of hemp flowers, hemp leaves, hemp roots, and hemp seed cakes.

In yet another embodiment, the mint leaves are selected from one or combination of peppermint or spearmint.

In yet another embodiment, the citrus is selected from one or combination of lemon, orange lime, or grapefruit.

In yet another embodiment, cannabis strain, ½ teaspoon calcium powder, ½ teaspoon magnesium powder, 1 teaspoon citrus, and 1 tablespoon mint leaves are mixed together in a coffee filter.

In yet another embodiment, the water is boiled at temperature such as 90° C., 95° C., 99° C., 100° C., 110° C., 115° C., 120° C., 125° C., 130° C., 135° C., 140° C., 145° C., 150° C., or similar, further preferably 95-120° C.

In yet another embodiment, a cannabinoid extraction method is described. The method includes the steps of providing one gram of cannabis strain; drying the strain and grinding the strain to obtain crude strain powder; providing ½ teaspoon each of calcium and magnesium, 1 teaspoon citrus and 1 tablespoon mint leaves; mixing the calcium, magnesium, citrus, mint leaves and strain in a 4″ x 4″ cheese cloth, keeping the cheese cloth in 1 cup water; boiling the water in a crockpot for couple of hours to extract a sticky substance “sap” on leaves and bud of cannabis.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a flow chart showing one potential embodiment of a natural cannabinoid extraction process of the present invention in accordance with the disclosed architecture; and

FIG. 2 illustrates a flow diagram showing another embodiment of a cannabinoid extraction process of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a cannabinoid extraction process that does not utilize butane or alcohol. There is also a long-felt need in the art for a cannabinoid extraction process that includes natural ingredients. Additionally, there is a long-felt need in the art for a cannabinoid extraction process that is easy to perform and is cost-effective. Moreover, there is a long-felt need in the art for a cannabinoid extraction process that eliminates mixing of butane or alcohol in CBD and THC. Further, there is a long-felt need in the art for a cannabinoid extraction process that provides the purest form of cannabinoid to consumers. Furthermore, there is a long-felt need in the art for a cannabinoid extraction process that can be used for both commercial and personal production of cannabis oils. Finally, there is a long-felt need in the art for a cannabinoid extraction process that offers a way to extract cannabinoids swiftly and easily for making cannabis edibles.

The present invention, in one exemplary embodiment, is a method of extracting cannabinoid from cannabis without using butane or alcohol. The method comprising the steps of providing one gram of cannabis strain; providing ½ teaspoon of calcium powder, ½ teaspoon of magnesium powder, 1 teaspoon of citrus, 1 tablespoon of mint leaves, and 1 cup of filtered water; mixing the calcium powder, magnesium powder, citrus and mint leaves; storing the mixture in a 4″ x 4″ cheese cloth piece; tying the cheese cloth piece using a hemp string; placing the cheese cloth piece like a tea bag in the filtered water placed inside a pressure cooker; boiling the water in the pressure cooker for 7 minutes; and extracting sap of leaves and bud of cannabis for providing THC and CBD.

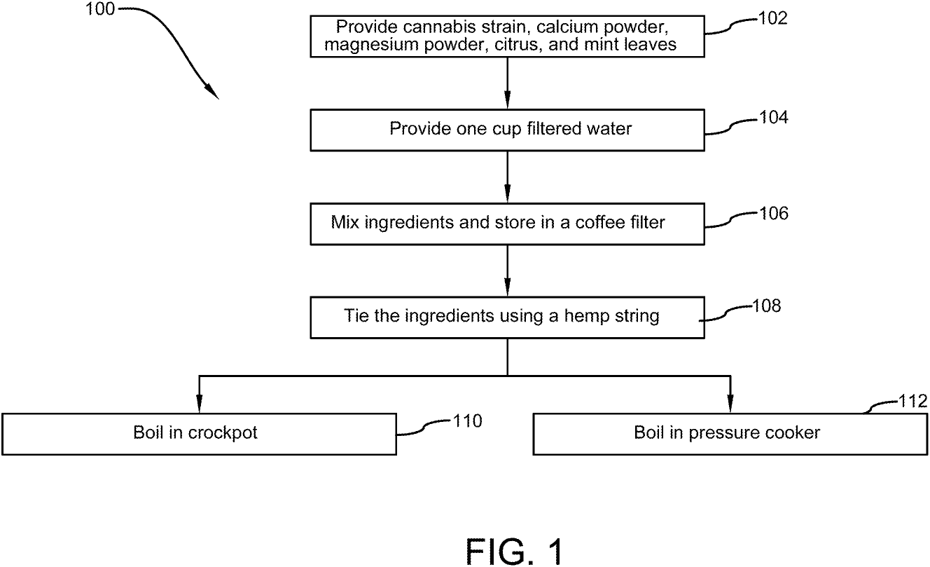

Referring initially to the drawings, FIG. 1 illustrates a flow chart showing one potential embodiment of natural cannabinoid extraction process of the present invention in accordance with the disclosed architecture. The cannabinoid extraction process 100 is natural and does not use butane or alcohol to extract CBD, TCH, and more. Further, the method is swift and can be performed without any professional equipment at home. During the process for one exemplary method for cannabinoid extraction, initially, one gram (or ¼ teaspoon) of cannabis strain, ½ teaspoon of calcium powder, ½ teaspoon of magnesium powder, 1 teaspoon citrus, 1 tablespoon (or 3 teaspoons) mint leaves are taken and mixed (Step 102). Thereafter, one cup (or 48 teaspoons) filter water is taken using a measuring cup (Step 104). Then, all the ingredients are placed in a coffee filter or, for example, a 4″ x 4″ cheese cloth piece, such as a tea bag (Step 106). After that, the ingredients are secured in the filter by tying closed the filter/cloth piece using a hemp string (Step 108). Finally, the filter and ingredients are placed in, for example, a crockpot containing the water. The water temperature is then raised to a boil in the crockpot and the filter with ingredients is retained in the boiling water for a couple of hours to create sap on the leaves and bud of cannabis (Step 110). Alternatively, the water and ingredients can be boiled in a pressure cooker for seven minutes and allowed to cool before use (Step 112). It is to be appreciated that 4.2 grams equals a teaspoon, but the nutrition facts usually rounds this number down to four grams. One tablespoon equals three teaspoons. One cup equals 16 tablespoons (or 48 teaspoons).

It should be noted that the process 100 of the present invention does not use any alcohol or butane and just uses heated water with natural ingredients to produce natural, organic, safe, and quick cannabinoid and cannabis edibles. For citrus, natural lemons, oranges, limes, or any other citrus fruit may be used. Citrus is used as an antidote to cannabis intoxication and helps to counteract the effect of THC/CBD.

Mint leaves can be of spearmint or peppermint and are used for enhancing stress relief and relaxation properties of cannabis. Mint also functions as an herbal supplement and improves health and vitality. The CBD or THC strain has preferably less than 0.5 percent THC and may be a pure CBD or hemp strain. The strain selected for the extraction process can be one or more of sativa, indica, or a combination of both.

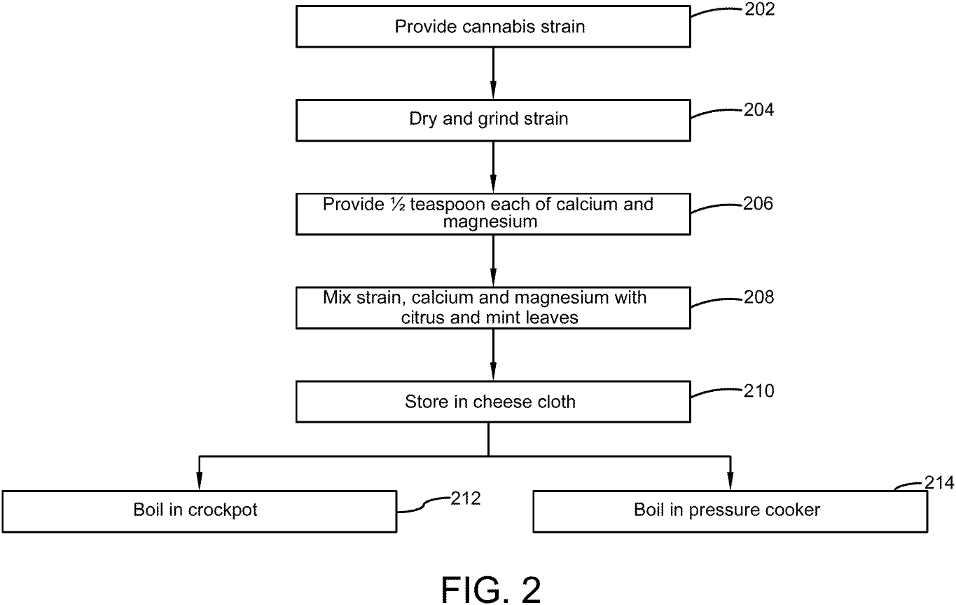

FIG. 2 illustrates a flow diagram showing another embodiment of cannabinoid extraction process of the present invention in accordance with the disclosed architecture. In the present embodiment, the extraction method that does not use butane or alcohol and uses natural ingredients. Initially, one gram of cannabis strain is provided (Step 202). Then, the strain is dried and grinded to obtain crude strain powder in which water is about 1.5% (Step 204). Thereafter, natural ingredients including ½ teaspoon each of calcium and magnesium is provided (Step 206) and mixed with 1 teaspoon citrus and 1 tablespoon mint leaves (Step 208).

All the ingredients, including the strain is mixed and can be tied in a 4″ x 4″ cheese cloth (Step 210). Then, the cheese cloth is kept in 1 cup water and is boiled in a crockpot for a couple of hours to extract a sticky substance “sap” on leaves and bud of cannabis (Step 212). Alternatively, for a quick process, the boiling is done in a pressure cooker for seven minutes to extract sticky substance “sap” on leaves and bud of cannabis (Step 214).

Calcium is used as an ingredient for improving osteoporosis curing properties of cannabidiol products and may be in the form of a natural source of calcium. Magnesium is also used for improving therapeutic properties of the cannabidiol.

In specific embodiments of the present invention, the yield of clean cannabis oil is not less than . percent, or not less than 85 wt. percent based on cumulative volume of the filter water. Further, in some embodiments, a filtering or recrystallization step is included as the final step that provides CBD having greater than 95% purity. In embodiments, the recrystallization provides CBD having at least 98% purity. It should be noted that during boiling of the ingredients in water, a pressure slightly higher than atmospheric pressure is maintained. Preferably, the pressure maintained is 1 ATM + from about 0.7 to about 0.9 ATM that allows the water to vaporize slowly and improve quality and quantity of sap and bud.

The cannabis strain can be any Cannabis spp plant material and can be purchased from a commercial or organic source. Cannabis spp is an annual broadleaf plant with a taproot and is capable of very rapid growth under ideal growing conditions. Cannabis can be used as an herb and is famous for containing the psychoactive component THC, but other distinct strains bred from this species have other unique biochemical compositions and uses. Plant material can be selected from one or more of inflorescence of male (staminate) plant, fruiting female (pistillate) plant, staminate flower, stamen, pollen grains, pistillate flower with bract, pistillate flower without bract, seeds, leaves, stalks, and roots.

In some embodiments, the water is boiled at temperature such as 90° C., 95° C., 99° C., 100° C., 110° C., 115° C., 120° C., 125° C., 130° C., 135° C., 140° C., 145° C., 150° C., or similar. In one exemplary range, the water is boiled at temperatures between 90-120° C.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “cannabinoid extraction process”, “natural cannabinoid extraction process”, “process”, and “cannabinoid extraction method” are interchangeable and refer to the natural cannabinoid extraction process 100 of the present invention.

Notwithstanding the forgoing, the natural cannabinoid extraction process 100 of the present invention can include any additional step or ingredient as is known in the art without affecting the overall concept of the invention, provided that it accomplishes the above-stated objectives. One of ordinary skill in the art will appreciate that the natural cannabinoid extraction process 100 as shown in the FIGS. are for illustrative purposes only, and that many other changes and modifications are well within the scope of the present disclosure. Any modification, equivalent replacement, or improvement made within the spirit and principle of the present invention shall fall into the protection scope of the present invention. The protection scope of the present invention is defined by the appended claims, and covers the equivalent changes of the claims.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A natural cannabinoid extraction method comprising the steps of:
combining a plurality of ingredients comprising a cannabis strain, a calcium powder, a magnesium powder, a citrus, and mint leaves;
mixing said plurality of ingredients;
placing said plurality of ingredients into a filter;
securing said filter in a closed position;
placing said filter and said plurality of ingredients into water;
boiling said water; and
creating sap extracted from said cannabis strain.

2. The method of claim 1, wherein said plurality of ingredients comprise a ratio generally of 1 part said cannabis strain, 2 parts said calcium powder, 2 parts said magnesium powder, 4 parts said citrus, and 12 parts said mint leaves.

3. The method of claim 1, wherein said plurality of ingredients comprise a ratio generally of ¼ teaspoon said cannabis strain, ½ teaspoon said calcium powder, ½ teaspoon said magnesium powder, 1 teaspoon said citrus, and 3 teaspoons said mint leaves.

4. The method of claim 2, wherein said filter is a coffee filter.

5. The method of claim 2, wherein said filter is a cheese cloth filter.

6. The method of claim 2, wherein said filter is a tea bag.

7. The method of claim 5, wherein said securing said filter comprises tying said filter closed with a hemp string.

8. The method of claim 3, wherein said water comprises a quantity of about 1 cup.

9. The method of claim 8, wherein said boiling said water comprises a temperature from about 90° C. to about 120° C.

10. The method of claim 9 further comprising a step of said boiling said water in a crockpot for a couple of hours.

11. The method of claim 9 further comprising a step of said boiling said water in a pressure cooker for several minutes.

12. The method of claim 1, wherein said mint leaves are selected from spearmint leaves and peppermint leaves.

13. The method of claim 12, wherein said citrus is selected from a group consisting of a natural lemon, a natural orange, and a natural lime.

14. The method of claim 1, wherein said cannabis strain is selected from sativa and indica.

15. A natural cannabinoid extraction method comprising the steps of:
combining a plurality of ingredients comprising a calcium powder, a magnesium powder, a citrus, and mint leaves;
adding a cannabis strain to said plurality of ingredients wherein said cannabis strain is dried and ground to create a crude cannabis strain powder;
mixing said plurality of ingredients and said cannabis strain;
placing said plurality of ingredients and said cannabis strain into a filter;
securing said filter in a closed position;
placing said filter, said plurality of ingredients, and said cannabis strain into water;
boiling said water; and
creating sap extracted from said cannabis strain.

16. The method of claim 15, wherein said plurality of ingredients and said cannabis strain comprise a ratio generally of 1 part said cannabis strain, 2 to 3 parts said calcium powder, 2 to 3 parts said magnesium powder, 4 to 5 parts said citrus, and 12 to 16 parts said mint leaves.

17. The method of claim 15, wherein said plurality of ingredients and said cannabis strain comprise a ratio generally of ¼ teaspoon said cannabis strain, ½ to ¾ teaspoon said calcium powder, ½ to ¾ teaspoon said magnesium powder, 1 to 1 ¼ teaspoons said citrus, and 3 to 4 teaspoons said mint leaves.

18. The method of claim 16, wherein said filter is a cheese cloth filter.

19. A natural cannabinoid extraction method comprising the steps of:
providing about 1.0 gram of a cannabis strain;
providing from about 2.0 grams to about 3.0 grams of a calcium powder;
providing from about 2.0 grams to about 3.0 grams of a magnesium powder;
providing from about 4.0 grams to about 5.0 grams of a citrus;
providing from about 12.0 grams to about 16.0 grams of a mint leaf;
mixing said cannabis strain, said calcium powder, said magnesium powder, said citrus, and said mint leaf into a mixture;
storing said mixture in a filter;
placing said filter and said mixture into boiling water for a duration; and extracting a sap for providing cannabinoid.

20. The method of claim 19, wherein said cannabis strain is selected from sativa and indica.
